(19) United States
(12) Patent Application Publication
(10) Pub. No.: US 2016/0374588 A1
(43) Pub. Date: Dec. 29, 2016

(54) MONITORING HYDRATION BASED ON GALVANIC SKIN RESPONSE

(71) Applicant: Microsoft Technology Licensing, LLC, Redmond, WA (US)

(72) Inventors: Farah Shariff, Kirkland, WA (US); Vinod L. Hingorani, Redmond, WA (US); Haithem Albadawi, Redmond, WA (US)

(21) Appl. No.: 14/749,124

(22) Filed: Jun. 24, 2015

(51) Int. Cl. A61B 5/053; A61B 5/00

(52) U.S. Cl. CPC A61B 5/0537; A61B 5/0533; A61B 5/4875; A61B 5/7475; A61B 5/721; A61B 5/746; A61B 5/7285; A61B 5/7264; A61B 5/681

(57) ABSTRACT

Examples are disclosed herein that are related to monitoring body hydration levels based on galvanic skin response measurements acquired by a wearable electronic device. One example provides a wearable electronic device including a sensor configured to measure a galvanic skin response, a logic device, and a storage device including instructions executable by the logic device to operate a hydration monitoring mode, acquire a plurality of measures of galvanic skin response over time, present data regarding the plurality of measures of galvanic skin response.

### BACKGROUND

Electronic devices may be configured to track and output information regarding physiological characteristics of a person, such as health and fitness data. Such information may be determined based, for example, upon biometric sensor data acquired by the computing device as the person performs various activities.

### SUMMARY

Examples are disclosed herein that are related to monitoring body hydration levels based on galvanic skin response measurements acquired by a wearable electronic device. One example provides a wearable electronic device comprising a sensor configured to measure a galvanic skin response, a logic device, and a storage device. The storage device comprises instructions executable by the logic device to operate a hydration monitoring mode, acquire a plurality of measures of galvanic skin response over time, and present data regarding the plurality of measures of galvanic skin response.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1A and 1B show an example wearable electronic device.

FIGS. 2A-C show example use scenarios for monitoring hydration via a galvanic skin response sensor of a wearable electronic device.

FIG. 3 shows an example galvanic skin response measurements and corresponding operation of a wearable electronic device.

FIG. 4 is a flowchart illustrating an example method of monitoring hydration based on galvanic skin response data.

FIG. 5 schematically illustrates an example sensory and logic system of a wearable electronic device.

### DETAILED DESCRIPTION

As mentioned above, electronic devices may monitor physiological characteristics via sensor data and provide feedback related to a person's health and fitness. For example, some wearable electronic devices may be configured to acquire and track various health and biometric data, including but not limited to heart rate, respiration rate, skin/body temperature, and calorie intake and expenditure, via sensor inputs.

Sensors on a wearable electronic device also may be used to detect electrical characteristics of skin. As such, examples are disclosed herein that relate to monitoring changes in body hydration levels via galvanic skin response sensor (GSR) data acquired via a GSR sensor on a wearable electronic device, and providing feedback regarding hydration levels.

Monitoring of and/or providing feedback related to hydration may be used in many scenarios. As one example, reminders or alerts to hydrate may be provided based upon hydration data acquired via a GSR sensor. As another example, a user may desire to continuously monitor and display hydration data while performing an activity, such as during exercise. As described below, different monitoring modes may be used for different activities, e.g. to provide power savings during when a physical activity level of a user is low while providing more granular data during periods of higher activity.

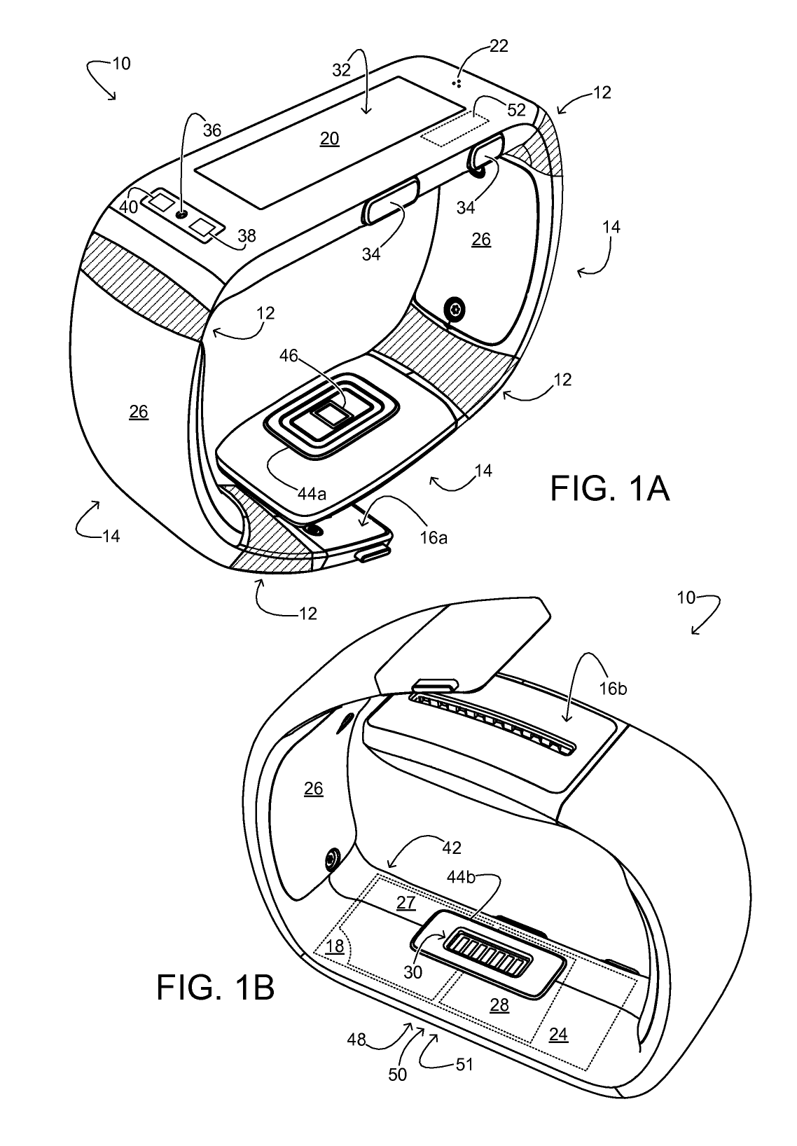

FIGS. 1A and 1B show aspects of an example sensory-and-logic system in the form of a wearable electronic device 10 that may be configured to operate in a hydration monitoring mode. The illustrated device is band-shaped and may be worn around a wrist. The wearable electronic device 10 includes at least four flexion regions 12 linking less flexible regions 14. The flexion regions 12 of the wearable electronic device 10 may be elastomeric in some examples. Fastening componentry 16a and 16b is arranged at both ends of the wearable electronic device 10. The flexion regions 12 and fastening componentry 16a and 16b enable the wearable electronic device 10 to be closed into a loop and to be worn on a user's wrist. In other examples, wearable electronic devices of a more elongate band shape may be worn around the user's bicep, waist, chest, ankle, leg, head, or other body part. The wearable electronic device 10, for example, may take the form of eye glasses, a head band, an arm-band, an ankle band, a chest strap, or an implantable device to be implanted in tissue.

The wearable electronic device 10 includes various functional components integrated into flexion regions 14. In particular, the wearable electronic device 10 includes a compute system 18, display 20, loudspeaker 22, communication suite 24, and various sensors. These components draw power from one or more energy-storage cells 26. A battery—e.g., a lithium ion battery—is one type of energy-storage cell suitable for this purpose. Examples of alternative energy-storage cells include super- and ultra-capacitors. In devices worn on the user's wrist, the energy-storage cells may be curved to fit the wrist, as shown in the drawings.

The energy-storage cells 26 may be replaceable and/or rechargeable. In some examples, recharge power may be provided through a universal serial bus (USB) port 30, which includes a magnetic latch to releasably secure a complementary USB connector. In other examples, the energy storage cells 26 may be recharged by wireless inductive or ambient-light charging. In still other examples, the wearable electronic device 10 may include electro-mechanical componentry to recharge the energy storage cells from the user's adventitious or purposeful body motion. For example, batteries or capacitors may be charged via an electromechanical generator integrated into the wearable electronic device 10. The generator may be turned by a mechanical armature that turns while the user is moving and wearing the wearable electronic device 10.

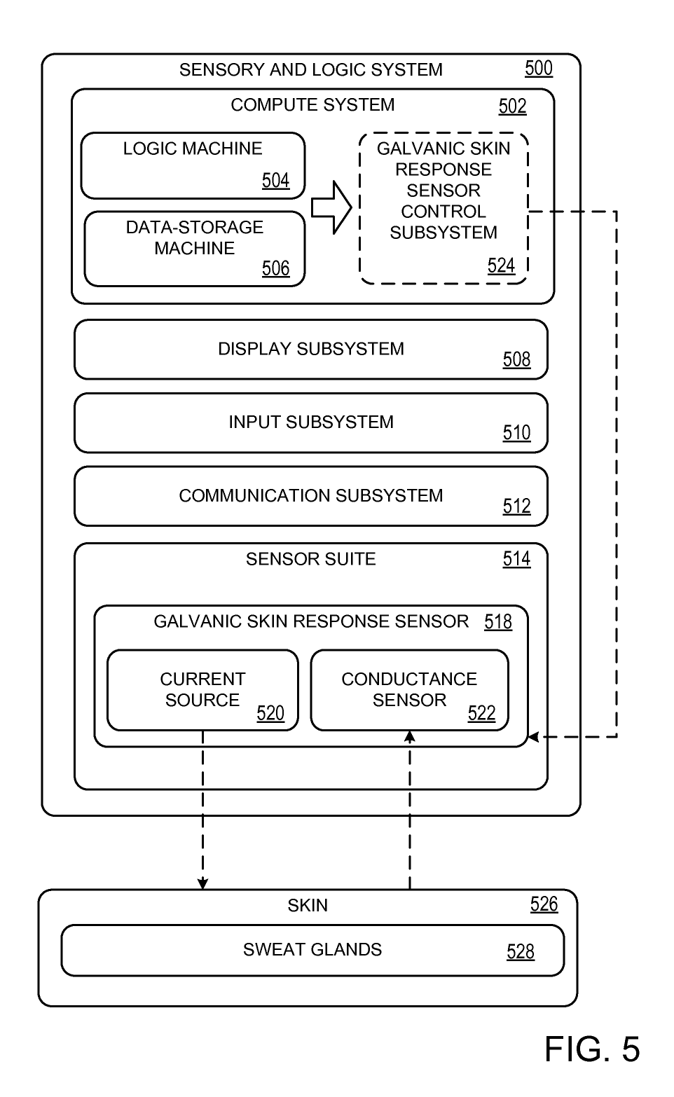

In the wearable electronic device 10, the compute system 18 is situated below the display 20 and operatively coupled to the display 20, along with the loudspeaker 22, the communication suite 24, and the various sensors and other components not depicted (e.g. haptic outputs, such as piezoelectric vibrators). The compute system 18 includes a data-storage machine 27 to hold data and instructions, and a logic machine 28 to execute the instructions. Aspects of the compute system 18 are described in further detail with reference to FIG. 5.

The display 20 may be any suitable type of display. In some configurations, a thin, low-power light emitting diode (LED) array or a liquid-crystal display (LCD) array may be used. An LCD array may be backlit in some implementations. In other implementations, a reflective LCD array, e.g., a liquid crystal on silicon (LCOS) array, may be frontlit via ambient light. A curved display may also be used. Further, active-matrix organic LED (AMOLED) displays or quantum dot displays may be used.

The communication suite 24 may include any appropriate wired or wireless communications componentry. In FIGS. 1A and 1B, the communication suite 24 includes USB port 30, which may be used for exchanging data between the wearable electronic device 10 and other computer systems, as well as providing recharge power. The communication suite 24 may further include two-way Bluetooth, Wi-Fi, cellular, near-field communication and/or other radios. In some examples, the communication suite may include an additional transceiver for optical, line-of-sight (e.g., infrared) communication.

In the wearable electronic device 10, touch-screen sensor 32 is coupled to display 20 and configured to receive touch input from the user. The touch-screen sensor 32 may be resistive, capacitive, or optically based. Pushbutton sensors may be used to detect the state of push buttons 34, which may include rockers. Input from the pushbutton sensors may be used to enact a home-key or on-off feature, control audio volume, turn the microphone on or off, etc.

FIGS. 1A and 1B show various other sensors of the wearable electronic device 10. Such sensors include microphone 36, visible-light sensor 38, ultraviolet sensor 40, and ambient temperature sensor 42. The microphone 36 provides input to the compute system 18 that may be used to measure the ambient sound level or receive voice commands from the wearer. Input from the visible-light sensor 38, ultraviolet senso 40r, and ambient temperature sensor 42 may be used to assess aspects of the wearer's environment—e.g., the temperature, overall lighting level, and whether the wearer is indoors or outdoors.

FIGS. 1A and 1B show a pair of contact sensor modules 44a and 44b, which contact the wearer's skin when the wearable electronic device 10 is worn. The contact sensor modules 44a and 44b may include independent or cooperating sensor elements, to provide a plurality of sensory functions. For example, the contact sensor modules 44a and 44b may provide an electrical resistance and/or capacitance sensory function responsive to the electrical resistance and/or capacitance of the wearer's skin, and thus may be configured to function as a GSR sensor. In the illustrated configuration, the separation between the two contact sensors provides a relatively long electrical path length, for more accurate measurement of skin resistance compared to a shorter path. Further, in some examples, a skin temperature sensor may be integrated into one or both of contact sensor modules 44a and 44b to provide measurement of the wearer's skin temperature.

The wearable electronic device 10 may also include motion sensing componentry, such as an accelerometer 48, gyroscope 50, and magnetometer 51. The accelerometer 48 and gyroscope 50 may furnish inertial and/or rotation rate data along three orthogonal axes as well as rotational data about the three axes, for a combined six degrees of freedom. This sensory data can be used to provide a pedometer/calorie-counting function, for example. Data from the accelerometer 48 and gyroscope 50 may be combined with geomagnetic data from the magnetometer 51 to further define the inertial and rotational data in terms of geographic orientation. The wearable electronic device 10 may also include a global positioning system (GPS) receiver 52 for determining the wearer's geographic location and/or velocity. In some configurations, the antenna of the GPS receiver 52 may be relatively flexible and extend into flexion regions 12.

The compute system 18, via the sensory functions described herein, is configured to acquire various forms of information about the wearer of the wearable electronic device 10. Such information must be acquired and used with utmost respect for the wearer's privacy. Accordingly, the sensory functions may be enacted subject to opt-in participation of the wearer. In implementations where personal data is collected on the wearable electronic device 10 and transmitted to a remote system for processing, that data may be anonymized In other examples, personal data may be confined to the wearable electronic device 10, and only non-personal, summary data is transmitted to the remote system.

As mentioned above, GSR data acquired by the contact sensor modules 44a and 44b may be used to determine hydration characteristics of the user. GSR may be defined as a change in one or more electrical properties of the skin, including but not limited to skin conductance, resistance, impedance, potential, admittance, and any suitable combinations thereof. GSR may behave as a function of sweat gland activity, due to the conductive properties of sweat. For example, more sweating may result in higher skin conductance (lower resistance/impedance), while relatively less sweating may result in lower skin conductance (higher resistance/impedance).

GSR data may be used to provide information on a user's possible hydration state in any suitable manner For example, GSR data may be used to track information indicative of possible changes in hydration state. In such examples, a baseline hydration level may first be set by the wearable electronic device 10 or by user input, and outputs may be provided based upon changes to this baseline hydration level as estimated using GSR data. In some examples, a baseline hydration level may be assumed to be a hydration level corresponding to a well-hydrated state. The setting of the baseline hydration level may be performed by user input, e.g. after consuming water or other hydrating fluids and at rest, or may automatically be set based upon a time of day at which a user is likely to be well-hydrated (e.g. at a time likely to be after a meal). In other examples, the monitoring of hydration may be initialized in any other suitable manner.

After the baseline hydration level is determined, GSR data may be monitored over time to estimate changes in the hydration level. For example, when the GSR output indicates a greater level of sweat, the estimated hydration level may be reduced at a relatively faster rate than when the GSR sensor indicates a lesser level of sweat.

When it is determined that a threshold level of hydration has been lost, the wearable electronic device 10 may output information alerting the user to this possible condition. Such information may be output in any suitable manner For example, information on the estimated hydration levels may be provided on an ongoing basis on a graphical user interface. In such examples, hydration levels may be output as a qualitative assessment of hydration level based on changes to the baseline. Such a qualitative output may include descriptive terms (e.g., “well-hydrated,” “dehydrated,” “high,” “low”), graphical representations (e.g., line graphs tracking hydration over time, bar or pie charts representing a comparison of estimated current hydration level to charts), and/or any other suitable visual output. The wearable electronic device 10 also may present alerts based upon estimated hydration state. Such alerts may include visual alerts, such as display of instructions or notifications (e.g., “drink more water”, color codes related to possible hydration states), as well as auditory outputs, haptic outputs, etc.

Instead of or in addition to tracking an estimated amount of hydration lost due to activity, warnings or alerts may be provided based upon threshold levels of GSR sensor outputs, possibly in combination with other sensor data. For example, a high detected skin temperature combined with GSR data that shows little sweat on the skin may trigger the output of an alert to cool down and/or hydrate.

The tracking of an estimated hydration level may be adjusted when a user rehydrates. The occurrence of rehydration may be determined in any suitable manner, including but not limited to user inputs. For example, when a user turns off a hydration alert, a user interface may allow the user to indicate that rehydration has occurred, and potentially how much fluid was consumed. In other examples, a pre-determined quantity of fluid may be assumed to have been consumed when an alert is cancelled.

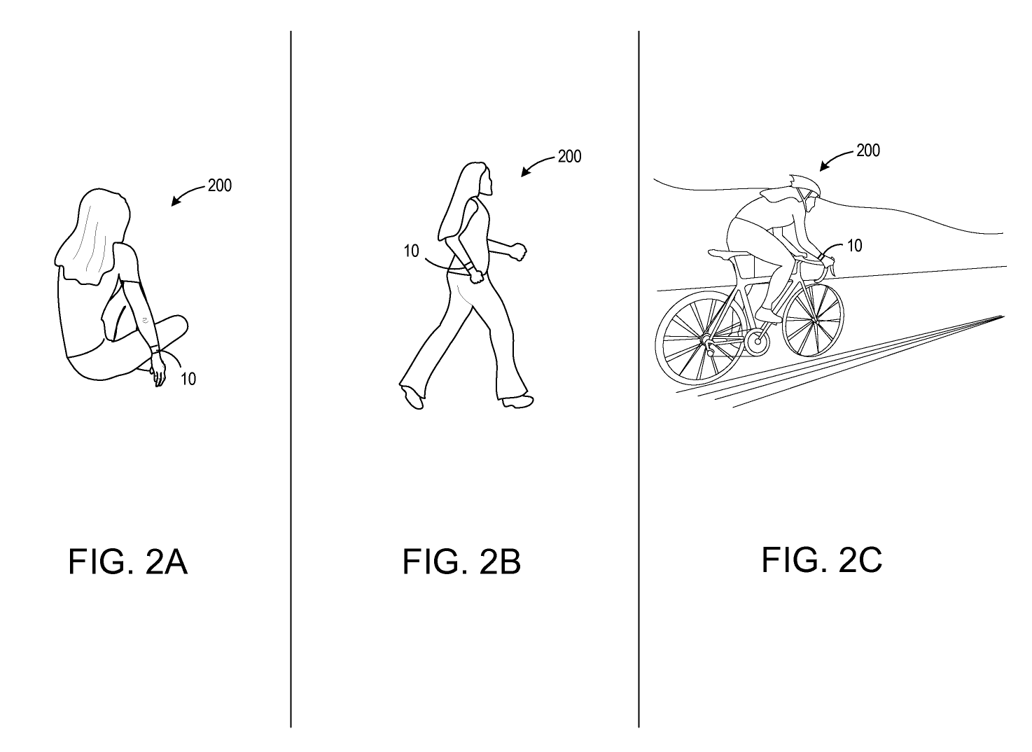

FIGS. 2A-C show various example scenarios in which the wearable electronic device 10 may be used to monitor hydration characteristics of a user 200. FIG. 2A depicts the user 200 at rest, FIG. 2B depicts the user 200 walking at a moderate pace, and FIG. 2C depicts the user 200 on a bicycle ride. In these examples, GSR data may indicate a lower level of sweat in the scenario of FIG. 2A than in 2B or 2C. As such, an estimated hydration level may change more slowly in the scenario of FIG. 2A than in FIGS. 2B-2C.

In some examples, the wearable electronic device 10 may have different hydration state monitoring modes that are triggered under different conditions. This may help to preserve computing resources when hydration states are likely to change slowly, while providing more granular data when hydration states are likely to change more quickly. The different modes may utilize different sensor data sampling rates, different analyses, and/or different outputs. For example, GSR data may be sampled at a higher sampling frequency in an activity mode, a lower frequency in a rest mode, and at an even lower frequency when in an idle/off mode. It will be understood that different hydration monitoring modes may utilize any suitable different operative states of the GSR sensor and/or other device components.

Any suitable trigger or triggers may be used to change hydration monitoring modes. For example, a user may make a user input to change the hydration monitoring mode from a rest mode to an activity mode before exercising, and then change back to rest mode once exercise is complete. Alternatively, such a trigger may be optional, as a hydration monitoring mode may be automatically entered whenever the wearable electronic device 10 is on.

The wearable electronic device 10 also may use sensor data to automatically trigger changes in operating modes. As an example, the GSR sensor may determine that the wearable electronic device 10 is removed from the wrist based upon various sensor data, including but not limited to data indicating an open circuit condition for the contact sensor modules 44a and 44b, and change from an active monitoring state to an idle/off state. Likewise, a change from an idle/off state to an active monitoring state may be triggered by detecting GSR data that exceeds a threshold noise level, indicating that the GSR contact modules are in contact with skin.

Further, different activity modes may be triggered based upon contextual sensor data indicating that a particular activity is being performed. Examples of such activities include, but are not limited to, rest, walking, jogging, running, biking, and swimming. Data from motion sensors (e.g. inertial measurement unit data, image sensor data), location sensors (e.g. GPS data), and other suitable data may be used to detect and distinguish such activities, as each activity may be distinguishable by such data. Thus, individual activities may have modes with different sensor sample rates and different associated analyses/outputs.

Contextual sensor data also may be used to disambiguate potentially ambiguous GSR data. As an example, where the wearable electronic device 10 detects a change in GSR indicating that the user's sweat output is decreasing, skin temperature data may be used to determine whether the GSR data is indicative of dehydration or of reduced exertion. Where a user's skin temperature increases beyond a threshold while the user's sweat output decreases, an alert may be output to remind the user to hydrate, whereas such a notice may not be output where the user's skin temperature decreases along with detected sweat output.

Different outputs and alerts may be provided in different operating modes. For example, referring to FIG. 2A, if the wearable electronic device 10 detects a change in GSR data indicating that the user's sweat activity has increased, but little or no change in movement or velocity, then the wearable electronic device may output information related to possible causes of increased agitation while at rest. As a more specific example, if the wearable electronic device 10 detects that the user's heart rate has also risen in conjunction with the increase in sweating, this may indicate that the user is under stress or nervous. As such, the wearable electronic device 10 may provide relevant output, such as feedback regarding qualitative stress levels, either independent of or related to hydration information.

In contrast, in the examples of FIG. 2B and 2C, other sensor data can be used by the wearable electronic device 10 to determine that the changes in GSR data correlate with activity or exercise. Thus, the wearable electronic device 10 may output input relating various exercise parameters to measured GSR and hydration characteristics, including but not limited to exercise performance in relation to hydration levels, recommendations to improve performance based on hydration, alerts to rehydrate, comparison data of current hydration levels compared to previous similar exercises, feedback regarding effort expended based on hydration data, time-dependent data showing hydration characteristics tracked throughout various stages of exercise, information on exercise recovery, and any other suitable hydration data related to user activity. It will be understood these examples are intended to be illustrative and not limiting in any manner

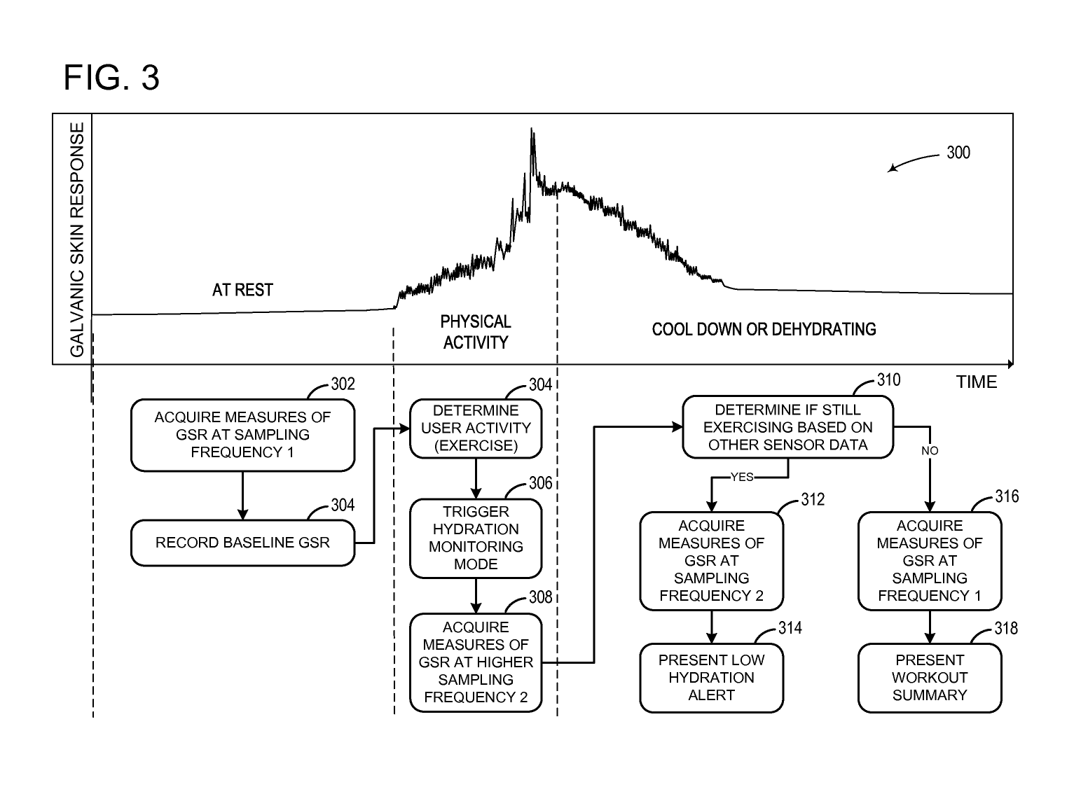

FIG. 3 shows an example plot 300 illustrating GSR data as measured by a GSR sensor of the wearable electronic device 10, and illustrates example changes in the operation of the GSR sensor based on the GSR data. Plot 300 indicates the magnitude of a signal output by the GSR sensor to a compute system over time. The GSR data may represent measured skin conductance, as one example, and/or any other suitable electrical property of the skin.

In the example of FIG. 3, a user wearing the wearable electronic device 10 may initially be at rest, as indicated at the left side of plot 300. During this state, the wearable electronic device 10 may acquire GSR measurements at a relatively lower first sampling frequency, as shown at 302. At 304, the wearable electronic device 10 may record baseline GSR data for the user, e.g. baseline skin conductance at an assumed baseline hydration level. At 304, the wearable electronic device 10 may determine that the user is no longer at rest and has started a form of physical activity, such as an exercise that has caused skin conductance to increase due to sweat loss. As described herein, a user activity may be determined via any suitable sensor data.

The determination of the start of a user activity triggers an active hydration monitoring mode, as shown at 306. In this mode, the wearable electronic device 10 may sample GSR data at a second, higher sampling frequency than the first sampling frequency, as shown at 308. In other instances, a determined user activity may cause the wearable electronic device 10 to sample GSR data at a lower sampling frequency, or maintain the sampling frequency, depending on the determined activities and potentially on other contextual data. In other examples, the wearable electronic device 10 may also consider remaining battery power, environmental sensor data, or any other suitable contextual information.

After a period of increasing GSR signal magnitude, the GSR signal then decreases for a period. Such a drop in GSR measurements may be a result of the user stopping exercising and cooling down, or may be a result of the user becoming progressively dehydrated as the user continues the exercise. Thus, at 310, the wearable electronic device may determine if the user is still exercising based on other sensor data, e.g. motion data and GPS data. If the wearable electronic device 10 determines that the user is still exercising, the wearable electronic device 10 may maintain the higher GSR data sampling frequency. Further, if it is determined that the user is becoming dehydrated, the wearable electronic device 10 may output a low hydration alert, as shown at 314. If the wearable electronic device 10 determines that the user is no longer exercising, the wearable electronic device 10 may return to measuring GSR at the lower sampling frequency in a rest mode. The wearable electronic device 10 may also present a workout summary, as shown at 318. Such a workout summary may include any suitable data regarding tracked hydration characteristics throughout the period of exercise.

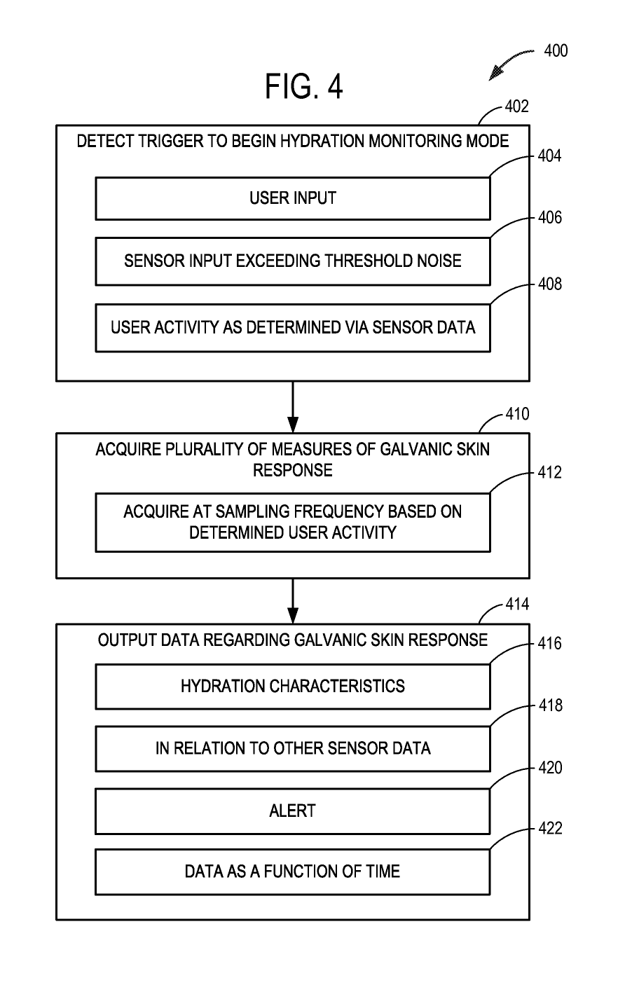

FIG. 4 illustrates an example method 400 for determining a hydration characteristic from GSR data. Method 400 comprises, at 402, detecting a trigger to begin a hydration monitoring mode. As described above, any suitable trigger may be used to initiate a hydration monitoring mode, including but not limited to user input, as shown at 404, a sensor input exceeding threshold noise, at 406, and/or a user activity as determined via sensor data, at 408. Method 400 further includes, at 410, acquiring a plurality of measures of GSR. Any suitable electrical characteristics of the skin may be measured, and the measurements may be acquired at any suitable sampling frequency. In some examples, a single sampling frequency may be used, while in other examples, the GSR data may be acquired at a sampling frequency based on a determined user activity, as indicated at 412. As described above, automatically changing the sampling frequency appropriately depending on the context may help to conserve power. It will be understood that any other suitable conditions may change the sampling frequency of the GSR sensor, including but not limited to power availability, user inputs, environmental conditions, etc.

Method 400 further includes, at 414, outputting data regarding the plurality of measures of GSR. The output may represent any suitable information determined from the GSR measurements, including hydration characteristics, as shown at 416. Information relating to hydration characteristics may include, but is not limited to, estimated body hydration level, estimated skin hydration level, estimated sweat loss, and estimated states of hydration (e.g. dehydration) based upon such estimates. Further, the GSR and hydration data may be presented in relation to other sensor data, as shown at 418. Other sensor data may include biometric data, such as heart rate, respiratory rate, and skin temperature, as well as motion sensor data (e.g. inertial motion sensor data, image sensor data) and data from other sensors that capture information related to the user. Other sensor data also may include environmental sensor data, such as ambient temperature and location.

The wearable electronic device also may output an alert, as indicated at 420. Such alerts may take any suitable form, including but not limited to visual, auditory, and/or haptic alerts. The alert may be configured to communicate any suitable information, such as notifications of different levels of dehydration and/or recommendations for the user to rehydrate.

Further, the data output at 414 may be output as a function of time to illustrate a time dependency, as shown at 422. The time-dependent data may represent, for example, tracked hydration data during a particular activity (e.g. a workout summary), hydration data compared or averaged across many occurrences of an activity (e.g. hydration levels during sleep for the past week), and/or any other suitable time-dependent information. It will be understood that any other useful feedback and output may be presented to the user regarding hydration characteristics based on GSR data.

In some embodiments, the methods and processes described herein may be tied to a computing system of one or more computing devices. In particular, such methods and processes may be implemented as a computer-application program or service, an application-programming interface (API), a library, and/or other computer-program product. FIGS. 1A and 1B show a non-limiting example of a computing system in the form of a sensory-and-logic system to implement the methods and processes described herein. As another non-limiting example, the disclosed methods and processes may also be enacted on other suitable sensory-and-logic systems, as shown schematically in FIG. 5.

FIG. 5 schematically shows an example sensory-and-logic system 500 that includes compute system 502 operatively coupled to a sensor suite 516. The compute system 502 includes a logic machine 504 and a data-storage machine 506. The compute system 502 is operatively coupled to a display subsystem 508, an input subsystem 510, a communication subsystem 512, and/or other components not shown in FIG. 5.

The logic machine 504 includes one or more physical devices configured to execute instructions. The logic machine 504 may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic machine 504 may include one or more processors configured to execute software instructions. Additionally or alternatively, the logic machine 504 may include one or more hardware or firmware logic machines configured to execute hardware or firmware instructions. Processors of the logic machine 504 may be single-core or multi-core, and the instructions executed thereon may be configured for sequential, parallel, and/or distributed processing. Individual components of the logic machine 504 optionally may be distributed among two or more separate devices, which may be remotely located and/or configured for coordinated processing. Aspects of the logic machine 504 may be virtualized and executed by remotely accessible, networked computing devices in a cloud-computing configuration.

The data-storage machine 506 includes one or more physical devices configured to hold instructions executable by the logic machine 504 to implement the methods and processes described herein. When such methods and processes are implemented, the state of the data-storage machine 506 may be transformed—e.g., to hold different data. The data-storage machine 506 may include removable and/or built-in devices. The data-storage machine 506 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. The data-storage machine 506 may include volatile, nonvolatile, dynamic, static, read/write, read-only, random-access, sequential-access, location-addressable, file-addressable, and/or content-addressable devices.

It will be appreciated that data-storage machine 506 includes one or more physical devices. However, aspects of the instructions described herein alternatively may be propagated by a communication medium (e.g., an electromagnetic signal, an optical signal, etc.) that is not held by a physical device for a finite duration.

Aspects of the logic machine 504 and the data-storage machine 506 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The term “program” may be used to describe an aspect of the compute system 502 implemented to perform a particular function. In some cases, a program may be instantiated via the logic machine 504 executing instructions held by the data-storage machine 506. It will be understood that different programs may be instantiated from the same application, service, code block, object, library, routine, API, function, etc. Likewise, the same program may be instantiated by different applications, services, code blocks, objects, routines, APIs, functions, etc. The term “program” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

The display subsystem 508 may be used to present a visual representation of data held by the data-storage machine 506. This visual representation may take the form of a graphical user interface (GUI). As the herein described methods and processes change the data held by the storage machine, and thus transform the state of the storage machine, the state of the display subsystem 508 may likewise be transformed to visually represent changes in the underlying data. The display subsystem 508 may include one or more display subsystem devices utilizing virtually any type of technology. Such display subsystem devices may be combined with the logic machine 504 and/or the data-storage machine 506 in a shared enclosure, or such display subsystem devices may be peripheral display subsystem devices. Display 20 of FIGS. 1A and 1B is an example of the display subsystem 510.

The input subsystem 510 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity. The touch-screen sensor 32 and push buttons 34 of FIGS. 1A and 1B are examples of the input subsystem 510.

The communication subsystem 512 may be configured to communicatively couple the compute system 500 to one or more other computing devices. The communication subsystem 512 may include wired and/or wireless communication devices compatible with one or more different communication protocols. As non-limiting examples, the communication subsystem 512 may be configured for communication via a wireless telephone network, a local- or wide-area network, and/or the Internet. The communication suite 24 of FIGS. 1A and 1B is an example of communication subsystem 512.

The sensor suite 514 may include one or more different sensors—e.g., a touch-screen sensor, push-button sensor, microphone, visible-light sensor, ultraviolet sensor, ambient-temperature sensor, contact sensors such as GSR sensor and skin temperature sensor, and/or GPS receiver—as described above with reference to FIGS. 1A and 1B. The s The GSR sensor is shown at 518. The GSR sensor 518 may include a current source 520 and a conductance sensor 522. The compute system 502 may include a GSR sensor control subsystem 524, which may be communicatively coupled to the logic machine 504 and the data-storage machine 506. The current source 520 and conductance sensor 522 may take the form of a pair of electrodes in contact with the skin, as described above. The current source 520 may be configured to pass a suitably minute amount of current through the skin 526 of the user from electrode to electrode, while the conductance sensor 522 may be configured to measure the direct current passing between the two electrodes. As mentioned above, GSR data may be measured as the variation in conductance as affected by the activity of sweat glands 528. It will be understood that GSR data may be represented by variations in any other suitable electrical property of the skin other than or in addition to conductance, including but not limited to resistance, impedance, potential, admittance, and/or any combinations thereof.

The GSR sensor 518 may receive raw signals from the conductance sensor 522, and may further process the raw signals to determine a hydration characteristic (e.g. sweat activity level, body hydration level). Control signals sent to the current source 520 and the conductance sensor 522 may be based on signals received from the conductance sensor 522, signals derived from the sensor suite 514, information stored in the data-storage machine 506, input received from the input subsystem 510, input received from the communication subsystem 512, etc.

Another example provides a wearable electronic device, comprising a sensor configured to measure a galvanic skin response, a logic device, and a storage device comprising instructions executable by the logic device to operate a hydration monitoring mode, acquire a plurality of measures of galvanic skin response over time, and present data regarding the plurality of measures of galvanic skin response. The instructions may additionally or alternatively be executable to present data by presenting data regarding a hydration characteristic based on the plurality of measures of galvanic skin response. The instructions may additionally or alternatively be executable to acquire other biometric data and present data regarding the galvanic skin response in relation to the other biometric data. The instructions may additionally or alternatively be executable to detect a trigger to enter the hydration monitoring mode, wherein the trigger comprises a user input. The instructions may additionally or alternatively be executable to detect a trigger to enter the hydration monitoring mode, wherein the trigger comprises a user activity as determined via sensor data. The instructions may additionally or alternatively be executable to detect a trigger to enter the hydration monitoring mode, wherein the trigger comprises a sensor input exceeding a threshold noise level. The instructions may additionally or alternatively be executable to present data by presenting an alert upon detecting a low body hydration level based on the plurality of measures of galvanic skin response. The instructions may additionally or alternatively be executable to acquire the plurality of measures of galvanic skin response at a sampling frequency based on a determined user activity.

Another example provides a wearable electronic device, comprising a sensor configured to measure a galvanic skin response, a logic device, and a storage device comprising instructions executable by the logic device to detect a trigger to begin a hydration monitoring mode, acquire a plurality of measures of galvanic skin response over time, determine a hydration characteristic based on the plurality of measures of galvanic skin response, and present data regarding the hydration characteristic. The instructions may additionally or alternatively be executable to present data regarding the hydration characteristic based on the plurality of measures of galvanic skin response. In this example, the trigger may additionally or alternatively include a user input, a user activity as determined via sensor data, and a sensor input exceeding a threshold noise level. The instructions may additionally or alternatively be executable to present data by presenting an alert upon detecting a low body hydration level based on the plurality of measures of galvanic skin response. The instructions may additionally or alternatively be executable to acquire the plurality of measures of galvanic skin response at a sampling frequency based on a determined user activity.

Another example provides, on a computing device comprising a sensor, a method, comprising detecting a trigger to begin a hydration monitoring mode, acquiring a plurality of measures of galvanic skin response over time, and outputting data regarding the plurality of measures of galvanic skin response. The method may additionally or alternatively include presenting data regarding a hydration characteristic based on the plurality of measures of galvanic skin response. The method may additionally or alternatively include acquiring the plurality of measures of galvanic skin response at a sampling frequency based on a determined user activity. Detecting the trigger may additionally or alternatively include detecting one or more of a user input and a sensor input exceeding a threshold noise. Detecting the trigger may additionally or alternatively include determining a user activity via sensor data.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A wearable electronic device, comprising:
a sensor configured to measure a galvanic skin response;
a logic device; and
a storage device comprising instructions executable by the logic device to
operate a hydration monitoring mode,
acquire a plurality of measures of galvanic skin response over time, and
present data regarding the plurality of measures of galvanic skin response.

2. The wearable electronic device of claim 1, wherein the instructions are executable to present data by presenting data regarding a hydration characteristic based on the plurality of measures of galvanic skin response.

3. The wearable electronic device of claim 1, wherein the instructions are further executable to acquire other biometric data and present data regarding the galvanic skin response in relation to the other biometric data.

4. The wearable electronic device of claim 1, wherein the instructions are further executable to detect a trigger to enter the hydration monitoring mode, and wherein the trigger comprises a user input.

5. The wearable electronic device of claim 1, wherein the instructions are further executable to detect a trigger to enter the hydration monitoring mode, and wherein the trigger comprises a user activity as determined via sensor data.

6. The wearable electronic device of claim 1, wherein the instructions are further executable to detect a trigger to enter the hydration monitoring mode, and wherein the trigger comprises a sensor input exceeding a threshold noise level.

7. The wearable electronic device of claim 1, wherein the instructions are executable to present data by presenting an alert upon detecting a low body hydration level based on the plurality of measures of galvanic skin response.

8. The wearable electronic device of claim 1, wherein the instructions are executable to acquire the plurality of measures of galvanic skin response at a sampling frequency based on a determined user activity.

9. A wearable electronic device, comprising:
a sensor configured to measure a galvanic skin response;
a logic device; and
a storage device comprising instructions executable by the logic device to
detect a trigger to begin a hydration monitoring mode,
acquire a plurality of measures of galvanic skin response over time,
determine a hydration characteristic based on the plurality of measures of galvanic skin response, and
present data regarding the hydration characteristic.

10. The wearable electronic device of claim 9, wherein the instructions are executable to present the data regarding the hydration characteristic as a graph.

11. The wearable electronic device of claim 9, wherein the trigger comprises a user input.

12. The wearable electronic device of claim 9, wherein the trigger comprises a user activity as determined via sensor data.

13. The wearable electronic device of claim 9, wherein the trigger comprises a sensor input exceeding a threshold noise level.

14. The wearable electronic device of claim 9, wherein the instructions are executable to present data by presenting an alert upon detecting a low body hydration level based on the plurality of measures of galvanic skin response.

15. The wearable electronic device of claim 9, wherein the instructions are executable to acquire the plurality of measures of galvanic skin response at a sampling frequency based on a determined user activity.

16. On a computing device comprising a sensor, a method, comprising:
detecting a trigger to begin a hydration monitoring mode;
acquiring a plurality of measures of galvanic skin response over time; and
outputting data regarding the plurality of measures of galvanic skin response.

17. The method of claim 16, further comprising presenting data regarding a hydration characteristic based on the plurality of measures of galvanic skin response.

18. The method of claim 16, further comprising acquiring the plurality of measures of galvanic skin response at a sampling frequency based on a determined user activity.

19. The method of claim 16, wherein detecting the trigger comprises detecting one or more of a user input and a sensor input exceeding a threshold noise.

20. The method of claim 16, wherein detecting the trigger comprises determining a user activity via sensor data.
